(19) United States
(12) Patent Application Publication
(10) Pub. No.: US 2014/0288418 A1
(43) Pub. Date: Sep. 25, 2014

(54) APPARATUS AND METHODS FOR PATHLENGTH MULTIPLEXING FOR ANGLE RESOLVED OPTICAL COHERENCE TOMOGRAPHY

(71) Applicant: Research Development Foundation, Carson City, NV (US)

(72) Inventors: Thomas MILNER, Austin, TX (US); Jordan DWELLE, Austin, TX (US); Biwei YIN, Austin, TX (US); Bingqing WANG, Austin, TX (US); Austin McELROY, Austin, TX (US); Grady RYLANDER, Round Rock, TX (US)

(73) Assignee: RESEARCH DEVELOPMENT FOUNDATION, Carson City, NV (US)

(21) Appl. No.: 14/215,303

(22) Filed: Mar. 17, 2014

Related U.S. Application Data
(60) Provisional application No. 61/787,840, filed on Mar. 15, 2013.

(51) Int. Cl. A61B 5/00; A61B 3/10; A61B 5/02

(52) U.S. Cl. CPC A61B 5/0066; A61B 5/02007; A61B 3/102; A61B 5/742
USPC 600/425

(57) ABSTRACT

Exemplary embodiments include an apparatus and method for performing angle-resolved imaging of scattering samples such as tissue, including the use of a pathlength multiplexing element.

### CROSS-REFERENCE TO RELATED APPLICATIONS

This application claims priority to U.S. Provisional Patent Application Ser. No. 61/787,840, filed Mar. 15, 2013, the contents of which are incorporated by reference herein.

### BACKGROUND INFORMATION

Optical coherence tomography (OCT) is routinely used in the imaging of biological tissue, including by ophthalmologists to record diagnostic retinal images. Important diagnostic features in recorded OCT retinal images include structural integrity, foveal contour and thickness for macular degeneration and retinal nerve fiber layer (RNFL) thickness for glaucoma. In addition to these diagnostic features, the optical scattering properties of RNFL may provide useful diagnostic information. Changes of optical scattering properties in cells undergoing apoptosis, largely due to intensified mitochondrial fission, have been observed in a number of studies. Using a Fourier microscopy imaging instrument, Pasternack et al. found that early cell apoptosis is accompanied by mitochondrial fission which results in more isotropic or large-angle light scattering. Chalut et al. designed an angle-resolved low coherence interferometry system to measure light-scattering changes of cells in early apoptotic stages which the authors suggested may involve mitochondrial fission. Recently, Ju et al., reported that mitochondria fission in differentiated retinal ganglion cell (RGC) cultures is induced in response to a glaucoma-like environment of elevated hydrostatic pressure. The observed structural changes in mitochondrial networks associated with neuro-degenerative diseases suggest that angular scattering properties of RNFL may provide diagnostic information for retinal diseases like glaucoma.

Recent studies suggest that the change of RNFL scattering properties in glaucomatous eyes results in decreased RNFL reflectance, which was found to be a sensitive, robust and early diagnostic glaucoma indicator. For example, Dwelle et al. investigated RNFL thickness, phase retardation, birefringence and other parameters and found that the earliest change associated with elevated intraocular pressure (IOP) in glaucomatous eyes of non-human primates is decreased RNFL reflectance. Liu et al. compared the performance of multiple glaucoma diagnostic indicators including RNFL thickness, reflectance, birefringence, and phase retardation, and identified RNFL reflectance as the best indicator to distinguish between control and glaucoma eyes and control and glaucoma-suspect eyes. Huang et al observed decreased RNFL reflectance prior to decreased thickness in glaucomatous retinas in human subjects. These observations suggest that measurement of RNFL scattering properties during OCT retinal imaging may provide a valuable and early diagnostic indicator of glaucoma.

Although various angle-resolved OCT designs have been reported for speckle reduction, no prior systems are known to measure RNFL angular scattering properties. Moreover, because the numerical aperture of light backscattered from the retina is limited by the pupil of the subject's eye and/or the aperture of the OCT instrument, angle-resolved measurements record variation within the limiting numerical aperture. Exemplary embodiments disclosed herein comprise a scattering-angle resolved OCT (SAR-OCT) apparatus and methods capable of measuring spatial variation in the angular distribution of RNFL backscattered light in retinal images.

### SUMMARY

Exemplary embodiments of the present disclosure include an apparatus and method for performing angle-resolved imaging of scattering samples such as tissue. Angle resolved imaging measurements can provide structural information about the sample that is not discernable with standard OCT or polarization sensitive OCT. Specifically, angle-resolved OCT or angle-resolved PS-OCT can provide information about the relative size of the scattering centers in the tissue including sub-cellular structures such as mitochondria. Exemplary embodiments comprise modifications and improvements over existing OCT systems for recording angle-resolved images of scattering samples.

Many diseases (e.g., neurodegenerative conditions such as Alzheimer's and Parkinson's) correspond to tissues with anomalous sized scattering centers. Angle resolved OCT allows direct recording of images corresponding to different incident-scattering angles of light on the tissue.

Exemplary embodiments include the use of a pathlength multiplexing element (PME). In certain exemplary embodiments, the PME can be designed to minimize light loss (reflection or absorption), maintain a uniform optical pathlength, and minimize the introduction of decreased resolution due to unbalanced optical dispersion. These features can be accomplished with proper optical design.

Certain embodiments include an apparatus comprising: an optical coherence tomography light source configured to emit a first wavelength; a polarizer configured to polarize the first wavelength; a splitter configured to direct the first wavelength emitted from the coherence tomography light source to a reference path and to a sample path; and a pathlength multiplexing element. In specific embodiments, the pathlength multiplexing element comprises a plurality of regions configured to direct the first wavelength at a plurality of angles in the sample path.

In particular embodiments, the plurality of regions of the pathlength multiplexing element comprise a first radial region and a second radial region. In specific embodiments, the plurality of regions of the pathlength multiplexing element comprises a first azimuthal region and a second azimuthal region. In certain embodiments, the plurality of regions comprises four azimuthal regions. In particular embodiments, the plurality of regions comprise six azimuthal regions. In certain embodiments, the plurality of regions of the pathlength multiplexing element comprise a first radial region, a second radial region, a first azimuthal region and a second azimuthal region. In specific embodiments, the plurality of regions comprises a first region configured as an aperture formed in a second region comprising glass. In particular embodiments, the plurality of regions each comprise different refractive indices. In certain embodiments, the optical coherence tomography light source is a swept-source laser, and in specific embodiments, the swept-source laser is configured to produce a wavelength of approximately 1060 nm with a 100 kHz sweep rate.

In particular embodiments, the sample path is configured to direct the first wavelength toward a retina. In certain embodiments, the sample path is configured to direct the first wavelength toward vascular tissue. Specific embodiments further comprise an electro-optic modulator between the polarizer and the pathlength multiplexing element. In particular embodiments, the reference path comprises a first polarization beam splitter for a horizontal channel and a second polarization beam splitter for a vertical channel.

Exemplary embodiments also include a method of imaging a sample site, where the method comprises: emitting a first wavelength from an optical coherence tomography light source; directing the first wavelength to a reference path and a photodetector; and directing the first wavelength to a sample path and through a pathlength multiplexing element to a sample site. In certain embodiments, the first wavelength passes through a first region of the pathlength multiplexing element and is directed to the sample site at a first angle, and the first wavelength passes through a second region of the pathlength multiplexing element and is directed to the sample site at a second angle. Specific embodiments also comprise reflecting the first wavelength from the sample site and through the first region of the pathlength multiplexing element to the photodetector; reflecting the first wavelength from the sample site and through the second region of the pathlength multiplexing element to the photodetector; and performing a comparison of the first wavelength reflected from the sample site to the first wavelength from the reference path.

Particular embodiments further comprise determining the size of an object in the sample site based on the comparison of the first wavelength reflected from the sample site to the first wavelength from the reference path. In specific embodiments, the object in the sample site is a sub-cellular structure. In certain embodiments, the object in the sample site comprises mitochondria. In certain embodiments, the sample site comprises a retina, and in other embodiments the sample site comprises a vascular wall.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The invention may be better understood by reference to one of these drawings in combination with the detailed description of specific embodiments presented herein.

FIG. 1A shows a schematic of an apparatus according to an exemplary embodiment.

FIG. 1B shows a schematic of an apparatus according to an exemplary embodiment.

FIG. 2 shows a schematic of a pathlength multiplexing element and a sample site.

FIG. 3 shows a schematic of a pathlength multiplexing element.

FIG. 4 shows images obtained from apparatus according to an exemplary embodiment.

FIG. 5 shows images obtained from apparatus according to an exemplary embodiment.

FIG. 6 shows a schematic of an apparatus according to an exemplary embodiment.

FIG. 7 shows a schematic of an apparatus according to an exemplary embodiment.

FIG. 8 shows a schematic of an apparatus according to an exemplary embodiment.

FIG. 9 shows a schematic of an apparatus according to an exemplary embodiment.

### DETAILED DESCRIPTION OF ILLUSTRATIVE EMBODIMENTS

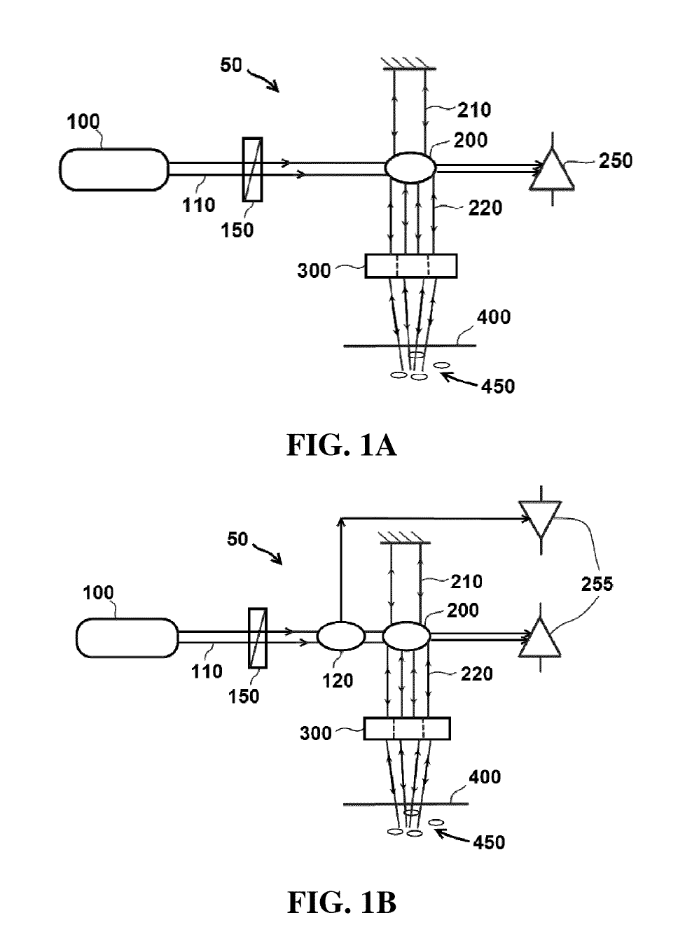

Referring now to FIG. 1A, a first embodiment of an apparatus 50 comprises an optical coherence tomography (OCT) light source 100 configured to emit a first wavelength 110, and a splitter 200 configured to direct first wavelength 110 to a reference path 210 and a sample path 220. Apparatus 50 can also comprise a polarizer 150 configured to polarize first wavelength 110 prior to first wavelength 110 reaching splitter 200. In addition, apparatus 50 can comprise a pathlength multiplexing element (PME) 300 that comprises a plurality of regions with different optical pathlengths configured to direct first wavelength 110 at a plurality of angles in sample path 220. Light propagating through each PME region is collimated and boundaries between regions are designed to absorb light so that any light that is incident on a region boundary, probability of either reflection and transmission events is minimized. In the embodiment shown in FIG. 1A, apparatus 50 also comprises a detector 250. PME 300 can incorporate discrete regions each with a spatial phase variation (e.g., quadratic) that provides a lensing or focusing effect on the light. In one embodiment, PME 300 may be configured as a lens with a central aperture. In another embodiment, PME 300 may comprise a plate in combination with a lens.

Referring now to FIG. 1B, another embodiment of apparatus 50 comprises components that are generally equivalent to those of FIG. 1A. However, the embodiment shown in FIG. 1B comprises a balanced detector 255 in lieu of detector 250 shown in the single-ended detector design of FIG. 1A. The embodiment of FIG. 1B also comprises a splitter 120 that directs a fraction of source light to the balanced detector.

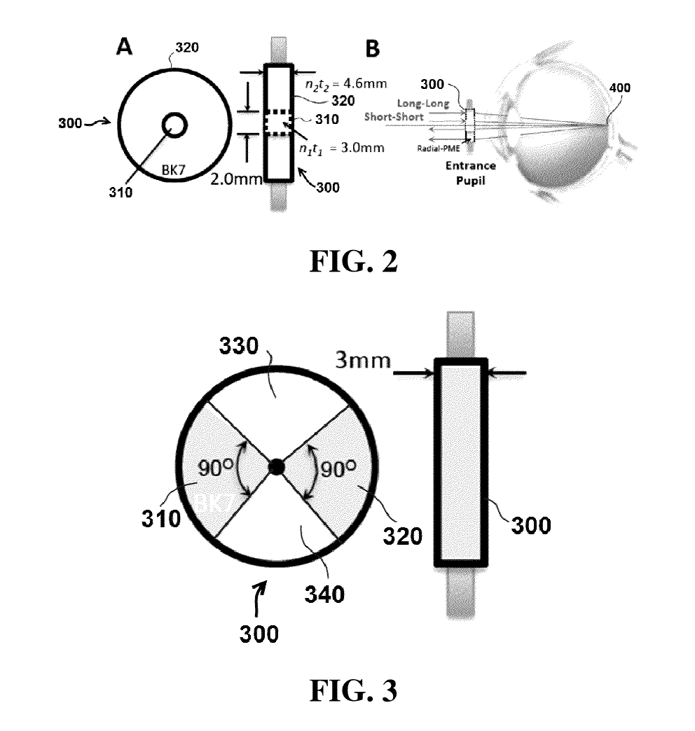

As shown in FIG. 2, in certain embodiments pathlength multiplexing element (PME) 300 may comprise a first radial region 310 and a second radial region 320. In certain embodiments, first radial region 310 may be an aperture formed in PME 300, and second region 320 may comprise glass. The boundary between region 310 and 320 can be coated with an absorbing material to minimize the probability of either reflection or transmission events. In other embodiments first radial region 310 may be a different material (e.g. a different type of glass or a plastic) than second radial region 320. In other embodiments the different regions may comprise the same material, but with different thicknesses.

During operation, apparatus 50 can be used to perform angle-resolved imaging of a sample 400, which may include scattering samples such as tissue. The PME is positioned in the pupil of the imaging system so that light propagating through each region is collimated and each lateral spatial position in the PME corresponds to an angle of light incident on and backscattered from the tissue sample. Angle resolved imaging measurements can provide structural information about sample 400 that is not discernable with standard OCT or polarization sensitive OCT (PS-OCT). Specifically, scattering angle-resolved OCT (SAR-OCT) or scattering angle-resolved PS-OCT can provide information about the relative size of scattering centers 450 in sample 400 including sub-cellular structures such as mitochondria. Many diseases correspond to tissues with anomalous sized scattering centers. SAR-OCT can allow direct recording of images corresponding to different incident- and back-scattering angles of light in tissue.

Specific exemplary embodiments can be configured to measure changes in the angular distribution of retinal nerve fiber layer (RNFL) backscattered light while simultaneously recording OCT retinal images. In such embodiments, SAR-OCT uses pathlength multiplexing to separate incident and backscattered light from the retina into discrete angular ranges. SAR-OCT is based on the principles of Fourier optics in which position of light in the pupil of the optical imaging system is directly proportional to the angle (with respect to the optical axis) of light incident on the RNFL. With SAR-OCT a PME can be positioned in a plane conjugate to the pupil so that light propagating through each region is collimated and optical pathlength of light returning from the RNFL is dependent on incident and backscattered angles. With the PME positioned in a plane conjugate to the pupil, each pair of PME regions can record a distinct SAR-OCT retinal sub-image that represents an angular range of light incident and backscattered from the RNFL.

For example, as shown in FIG. 2, PME 300 may comprise a first radial region 310 and a second radial region 320 configured to direct light at different angles to a patient's pupil. In the embodiment shown, region 310 is a shorter path and consists of an air-space inner circular aperture with a diameter of 2.0 mm and an optical thickness, n₁t₁=3.0 mm. Region 320 is a longer path and consists of a BK7 glass annular aperture with optical thickness, n₂t₂=4.6 mm. In certain embodiments, the surface separating region 310 from region 320 is absorbing to minimize the probability of either light reflection or transmission events.

In still other embodiments, PME 300 may comprise different azimuthal regions. For example, as shown in FIG. 3, PME 300 may comprise four separate regions 310, 320, 330, and 340 that each occupies a 90 degree sector. In still other embodiments, PME may comprise a different number of azimuthal regions, including for example, two, three, five, six or more different regions that comprise 180, 120, 72, or 60 degrees.

In exemplary embodiments, PME 300 can be used with light source 100 configured as a swept-source (1060±30 nm) laser with a 100 KHz sweep rate. In particular embodiments, system 50 can be used to record peri-papillary radial scans at 100,000 A-Lines per second. In one particular embodiment, PME 300 can be positioned in a plane conjugate to pupil 400 so that region 310 records a SAR-OCT sub-image of the retina for light incident or backscattered at small angles (e.g., less than 4.8°). Light passing through region 320 can record a SAR-OCT sub-image of the retina for light incident or backscattered at larger angles (e.g., between 4.8° and 13.8°). When SAR-OCT light enters the patient's pupil and backscatters from the RNFL, four incident-backscattered light paths through PME 300 can be recognized: (1) short-short—SAR-OCT light incident on pupil 400 propagates through region 310 (low angle), backscatters from the RNFL at a low angle and returns through region 310 with a composite pathlength of 2n₁t₁; (2) short-long—SAR-OCT light incident on pupil 400 propagates through region 310 (low angle), backscatters from the RNFL at a high angle and returns through region 320 with a composite pathlength of n₁t₁+n₂t₂; (3) long-short—SAR-OCT light incident on pupil propagates through 320 (high angle), backscatters from the RNFL at a low angle and returns through 310 with a composite pathlength of n₁t₁+n₂t₂; (4) long-long—SAR-OCT light incident on pupil 400 propagates through 320 (high angle), backscatters from the RNFL at a high angle and returns through region 320 with a composite pathlength of 2n₂t₂.

As described above, the optical pathlength of two of the four light paths are degenerate: these include, short-long and long-short paths. Accordingly, SAR-OCT data consists of three retinal sub-images corresponding to: (1) low-angle incident/low-angle backscattered (short-short with optical pathlength of 2n₁t₁); (2) degenerate paths (short-long and long-short with an optical pathlength of n₁t₁+n₂t₂); and (3) high-angle incident/high-angle backscattered (long-long with optical pathlength of 2n₂t₂).

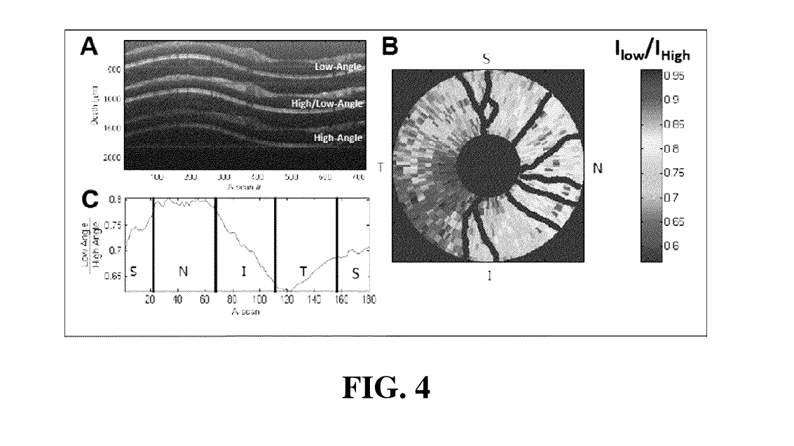

PME 300 can be used to record SAR-OCT retinal images, including those of a healthy human subject as shown in FIG. 4. As shown in section A, three SAR-OCT retinal sub-images corresponding to a ring scan with a diameter of 4.4 mm represent: (1) low-angle incident/low-angle backscattered (top scan); (2) low/high-angle degenerate paths (middle scan); (3) high-angle incident/high-angle backscattered (bottom scan). In certain embodiments, the images can be combined (i.e. weighted average) to obtain an image with reduced speckle as known in the art. Section B shows peripapillary variation of low-to-high angle RNFL backscattering anisotropy (I_{Low}/I_{High}). Section C shows a retinal map of low-to-high angle RNFL backscattering anisotropy (I_{Low}/I_{High}).

In one embodiment, a retinal map of the low-to-high angle RNFL backscattering anisotropy (I_{Low}/I_{High}) was computed by recording ten peri-papillary ring-scans centered on the optic nervehead with diameters ranging between 1.25-4.44 mm. To determine scattering properties of RGC axons, the RNFL was first segmented from each of the three SAR-OCT retinal sub-images. Relative strength of RNFL backscattering at small angle (less than 4.8°) and large angles (between 4.8° and 13.8°) was determined by correcting for two effects in low- and high-angle retinal sub-images: (1) spatial variation of SAR-OCT beam intensity in each area in PME 300; and (2) roll-off of the source coherence function with increased imaging depth.

After correction, low-to-high angle RNFL backscattering anisotropy (I_{Low}/I_{High}) can be computed for each of the ten peri-papillary ring scans and plotted over the imaged retinal area (shown in FIG. 4B). In this embodiment, low-to-high angle RNFL backscattering anisotropy (I_{Low}/I_{High}) was averaged over the ten ring scans to give the peri-papillary variation around the optic nervehead (shown in FIG. 4C). As noted in the figures, low-to-high angle RNFL backscattering anisotropy (I_{Low}/I_{High}) is smallest (largest) in the temporal (nasal) quadrant. SAR-OCT results suggest that for this healthy human subject, RGC axonal scattering structures (e.g., mitochondria networks) in the temporal (nasal) quadrant backscatter incident light at relatively larger (smaller) angles compared to those structures in the superior and inferior quadrants. RGC axons in normal human subjects are known to have smallest diameter in the temporal quadrant, so that angle of RNFL backscattered light in this region is expected to be larger and is consistent with low-to-high angle RNFL backscattering anisotropy (I_{Low}/I_{High}) determined from SAR-OCT.

In summary, PME 300 can be utilized in an ophthalmologic OCT imaging system comprising a swept-source (1060±30 nm) laser as a light source. SAR-OCT retinal images recorded from a healthy human subject suggest that low-to-high angle RNFL backscattering anisotropy (I_{Low}/I_{High}) varies with position around the optic nervehead. RGC axonal structures in the temporal (nasal) quadrant backscatter light at larger (smaller) angles compared to superior and inferior quadrants.

In certain embodiments, a scattering angle resolved polarization sensitive OCT (SAR-PSOCT) system may be used to measure both polarimetric and angular changes in RNFL backscattered light associated with RGC dysfunction. Exemplary embodiments of polarization-sensitive OCT (PSOCT) systems may be used to record images of RNFL thickness, phase retardation and birefringence in primate and human eyes. Addition of polarization sensitivity to SAR-OCT can enhance the ability to detect RGC dysfunction due to disruptions in the mitochondrial fusion/fission cycle and detect pre-perimetric glaucoma at an earlier stage.

Polarization state of light backscattered from RGC axons is dependent on many factors including axonal membranes, neurotubules and mitochondrial networks. The anisotropic structure of RGC axons gives rise to form-birefringence that originates primarily in neurotubules. It is believed that the state of the mitochondrial network (fusion/fission) in RGC axons can impact the polarization state of RNFL backscattered light. When mitochondria are fused together, light polarized parallel to the long axis of the mitochondrial network scatters differently than perpendicular oscillations and introduces a polarimetric-angular scattering anisotropy. In the fission state, mitochondria act more as independent scatterers so that polarimetric-angular scattering anisotropy is expected to be less. To investigate the utility of SAR-PSOCT instrumentation to detect RGC dysfunction, a computational model of RGC axons' scattering properties was constructed to investigate the polarimetric and angular variation of RNFL backscattered light.

The discrete dipole approximation (DDA) is an established computational method that may be applied to compute the polarimetric-angular anisotropy of backscattered light from cells. The DDA was applied to compute the polarimetric-angular variation of backscattered light from RGC axons with mitochondria in the fission and fusion states. In this model, an RGC axon is represented as an array of polarizable discrete dipoles by specifying refractive indices of RGC cytoplasm (1.36), mitochondria (1.43), and neurotubules (1.50). Following Maxwell's equations, Stokes vector of light backscattered from an RGC axon is given by solution to a large system of linear equations. To investigate differences in polarimetric-angular variation of backscattered light from RGC axons with mitochondria in fusion and fission states, two model RGC axons can be constructed. In both RGC axons, axonal segment length (2 μm), axon radius (0.7 μm), number of mitochondria (3), and number of neurotubules (100) are equal.

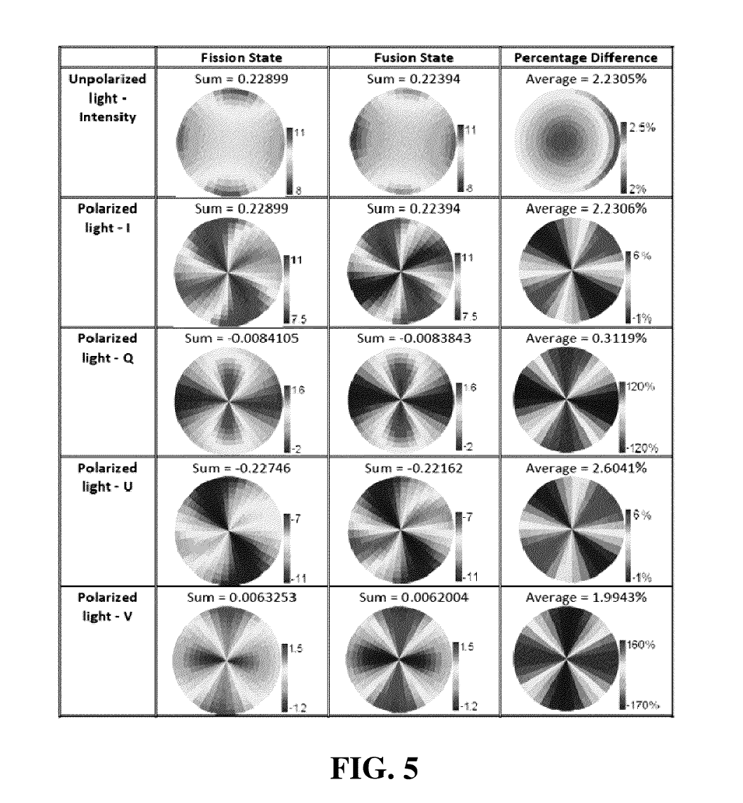

In the fission RGC axon, three mitochondria are separated spatially and each has a spherical shape (0.4 μm diameter). In the fusion RGC axon, three mitochondria contact each other end-to-end and each has an ellipsoidal shape (0.3, 0.3, and 0.8 μm diameters). Two states of incident light can be considered: (a) unpolarized; and (b) linearly-polarized light at 45 degrees to the axonal axis. Applying the DDA, Stoke's parameters (I, Q, U, and V) can be computed of backscattered light (±7 degrees) from RGC axons in the fission and fusion states, as shown in FIG. 5. When unpolarized light is incident on RGC axons, intensity of backscattered light from RGCs in the fission state is increased at high angles compared to the fusion state (FIG. 5, top row). When linearly polarized light at 45 degrees is incident on RGC axons, a significant polarimetric-angular anisotropy in backscattered light is observed for both fission and fusion states (FIG. 5, bottom four rows). As noted in the figure, FIG. 5 shows DDA calculation of the polarimetric-angular anisotropy of light backscattered from RGC axons (±7°) with mitochondrial networks in fission (left column) and fusion (middle column) states. The percentage difference is shown in the right column, and the top row is the incident unpolarized light. The bottom four rows are Stokes parameters (I, Q, U, V) of light backscattered from RGC axons for incident linear polarized light at 45° to RGC axons.

Moreover, significant differences (FIG. 5, rightmost column) between fission and fusion states are observed for all four Stokes parameters (I, Q, U, and V). Because simulated RGCs are 2.0×0.7 μm—a small fraction of actual RNFL axons—computed percentage differences (rightmost column) are substantially smaller than expected values measured from in-vivo retina.

In summary, DDA simulations suggest significant polarimetric-angular anisotropy exists in backscattered light from RGC axons with mitochondria in fusion and fission states. Exemplary embodiments of SAR-PSOCT apparatus can be used to provide an objective means to measure of RNFL backscattered anisotropy so that RGC dysfunction may be detected at the early stages in patients at risk for pre-perimetric glaucoma so that appropriate lifestyle changes or neuroprotective therapies may be administered to prevent vision loss.

In still other embodiments, PME 300 may comprise azimuthal regions, instead of (or in addition) radial regions. DDA simulation results suggest that backscattered light from

RGC axons with mitochondria in fusion and fission states can have a significant polarimetric-angular anisotropy. Because a single PME that incorporates two radial and two azimuthal regions would give ten retinal sub-images, a swept-source laser is required that supports a scan depth of 15-20 mm corresponding to a scan depth of 1.5-2.0 mm.

As an example embodiment, to detect the polarimetric-angular anisotropy of RNFL backscattered light observed in DDA simulations, an azimuthal PME with two regions can be utilized as shown in FIG. 3. Sector design of the azimuthal PME is motivated in part by the polarimetric-angular anisotropy observed in DDA simulations of light backscattered from RGC axons containing mitochondria in fission and fusion states FIG. 5. In the embodiment shown, azimuthal PME 300 can be constructed using two 3 mm thick 90-degree angular sectors constructed from BK7 glass. In a specific embodiment, the outer surfaces of the two glass angular sectors can be fastened with epoxy to a 25 mm stainless-steel ring, while the inner surfaces of can be fastened to a 1 mm ring.

When positioned in the SAR-PSOCT system, azimuthal PME 300 can provide three retinal sub-images: (1) vertical incident/vertical backscattered (short-short path); (2) degenerate vertical/horizontal paths (medium length path); and (3) horizontal incident/horizontal backscattered (long-long path). Azimuthal PME 300 can allow objective measurement of the RNFL backscattering anisotropy (I_{H}/I_{V}) and provide sensitive detection of differences in polarization-angular anisotropy of backscattered light from RGC axons with mitochondria in fission and fusion states.

### EXAMPLE 1

### SAR-PSOCT Retinal Imaging System

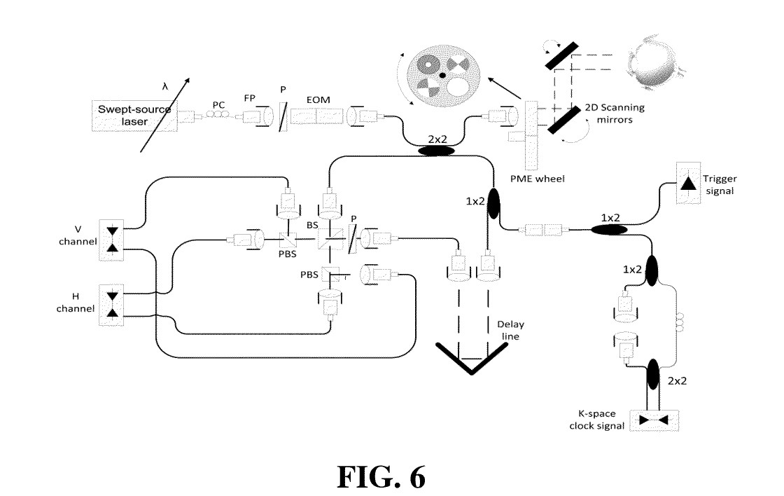

Referring now to FIG. 6 an SAR-PSOCT instrument uses a swept-source laser (1060±30 nm) with a 100 KHz sweep rate. An electro-optic modulator (EOM) sets the polarization state input into a two-beam fiber interferometer. Light in the fiber interferometer is split into sample and reference paths. Light in the sample path is collimated and propagates through either the radial or azimuthal PME mounted on a motorized wheel. The PME wheel contains radial and azimuthal elements. Two azimuthal PMEs oriented at 45 degrees with respect to each other are utilized to probe all directions of

RGC axonal anisotropy. The PME and scanning optics are mounted in a standard slit lamp for patient imaging. Light in the reference path is split into a fiber k-space clock and a trigger photodiode for SAR-PSOCT signal acquisition. Light returning from the patient's retina and reference path recombine in the beam splitter (BS) and are split into horizontal (H) and vertical (V) channels using polarization beam splitters (PBS). SAR-PSOCT signals are input into a high-speed analog-to-digital converter (500 MS/s) and digital data stored to a hard disc drive. SAR-PSOCT data recorded using the radial PME consists of three polarization dependent retinal sub-images corresponding to low-angle, high/low-angle and high-angle scattering. SAR-PSOCT data recorded using azimuthal PMEs consists of three polarization dependent retinal sub-images corresponding to horizontal, horizontal/vertical and vertical scattering angles. For either radial or azimuthal PMEs, polarization sensitivity of each SAR-PSOCT retinal sub-image data gives light amplitudes polarized parallel (L_{∥}) and perpendicular (I_{⊥}) to RGC axons and their relative phase [φ=arg(I_{∥})−arg(I_{⊥})].

During operation SAR-PSOCT instrumentation records polarization-sensitive and angular-resolved OCT sub-images of the RNFL for radial and azimuthal PMEs. Prior to recording a retinal image, a pre-scan can be recorded to set the incident polarization state relative to the local optical axis of the nerve fiber layer. The pre-scan can include a number of retinal locations (at least one in each of the four quadrants) and allow real-time specification of a single incident polarization state for each retinal location that provides equal light amplitudes parallel (I_{∥}) and perpendicular (I_{⊥}) to the axonal axis. By reducing the number of incident polarization states, a reduction in acquisition time is realized so that retinal images may be acquired in less than one second. After completing the pre-scan and specification of incident polarization states, retinal sub-images will be recorded for radial and azimuthal PMEs. Two SAR-PSOCT sub-images will be recorded for two azimuthal PMEs with a relative orientation of 45 degrees. The RNFL will be segmented in each of the SAR-PSOCT sub-images for radial and azimuthal PMEs and corrected for two effects: (1) spatial variation of SAR-OCT beam intensity in each PME area; and (2) roll-off of the source coherence function [Γ(n_{g}cτ)] with increasing SAR-PSOCT imaging depth. In addition to standard maps of RNFL thickness and reflectivity, maps of twelve new RNFL backscattering anisotropies are constructed (Table 1).

| Radial PME | | Azimuthal PME | |
|---|---|---|---|
| Low-High | I_{Low}/I_{High} | Azimuthal | I_{H}/I_{V} |
| Scattering | | Scattering | |
| Anisotropy | | Anisotropy | |
| Low-angle | I_{Low, ∥}/I_{Low, ⊥} | Horizontal | I_{H, ∥}/I_{H, ⊥} |
| Polarimetric | | Polarimetric | |
| Anisotropy | | Anisotropy | |
| High-angle | I_{High, ∥}/I_{High, ⊥} | Vertical | I_{V, ∥}/I_{V, ⊥} |
| Polarimetric | | Polarimetric | |
| Anisotropy | | Anisotropy | |
| High/Low-Angle | I_{Low, ∥}/I_{High, ∥}, | Horizontal/Vertical | I_{H, ∥}/I_{V, ∥}, |
| Polarimetric | I_{Low, ⊥}/I_{High, ∥} | Polarimetric | I_{V, ∥}/I_{V, ⊥} |
| Anisotropy | | Anisotropy | |
| High/Low-Angle | φ_{Low}-φ_{High} | Horizontal/Vertical | φ_{H}-φ_{V} |
| Retardation | | Phase-retardation | |

Table 1: RNFL backscattering anisotropies derived from SAR-PSOCT using radial and azimuthal PMEs.

For radial-PME SAR-PSOCT sub-images: I is the corrected RNFL intensity with subscripts denoting high-angle (High), low-angle (Low), polarized parallel to the nerve fiber (μ), polarized perpendicular to the nerve fiber (⊥), and φ is phase retardation [φ=arg(I_{∥})−arg(I_{⊥})]. In azimuthal PME SAR-PSOCT sub-images: I is the corrected RNFL intensity with subscripts denoting horizontal (H), vertical (V), polarized parallel to the nerve fiber (∥), polarized perpendicular to the nerve fiber (⊥), and φ is phase retardation [φ=arg(I_{∥})−arg(I_{⊥})]. Operation of the SAR-PS OCT instrument with radial and azimuthal PMEs is validated with form biregrigent phantoms known in the art.

### EXAMPLE 2

### SAR-IVOCT Intravascular Imaging System

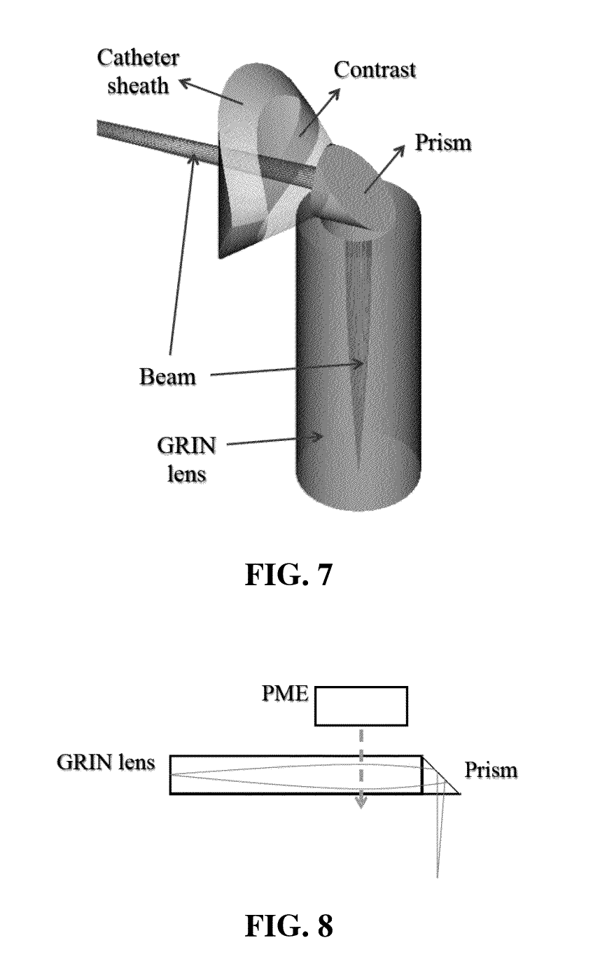

In certain embodiments, a scattering angle resolved intravascular OCT (SAR-IVOCT) apparatus and system can be used to provide non-invasive high resolution imaging and measurements of the reflected light from tissue discontinuities in intravascular applications. In certain embodiments, the system may comprise an axial resolution of 10-20 μm, a lateral resolution of 20-40 μm, and tissue penetration depth of 1.5-2.0 mm. As shown in FIG. 7, the apparatus may comprise a single-mode optical fiber, GRIN lens, glass prism, and a polymer sheath that is filled with contrast.

As shown in FIG. 8 a pathlength multiplexing element (PME) can be incorporated into the catheter to investigate scattering properties of structures within the vascular wall, via (SAR-OCT) with the light separated into discrete angular ranges.

In certain embodiments, data obtained from angle-resolved OCT systems may be used to distinguish between different cell types (including for example, M1 and M2 macrophages) based on differences in the high-high, low-low, and high-low incident/reflected signals.

### EXAMPLE 3

### SAR-IVOCT Intravascular Imaging System

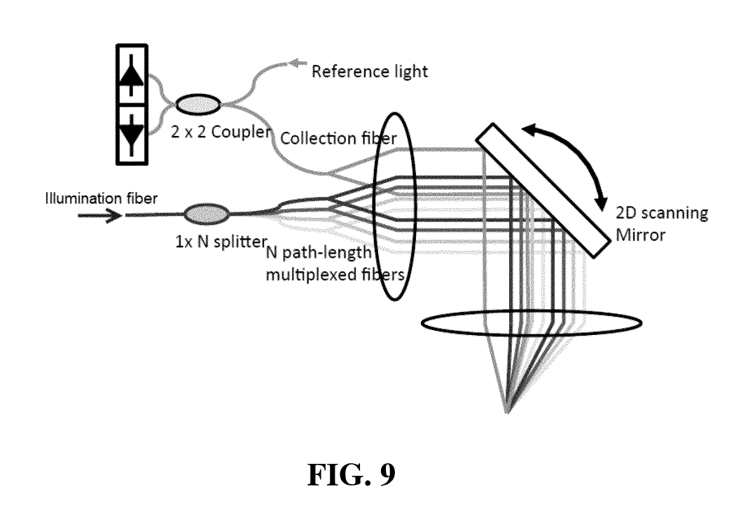

Referring now to FIG. 9, a scattering angle resolved intravascular OCT (SAR-IVOCT) apparatus and system comprises components equivalent to those in previously-described embodiments. This embodiment, however, also comprises a 1×N splitter in the illumination path between the illumination fiber and a plurality of N path-length multiplexed fibers that are directed to a 2-D scanning mirror. This configuration provides multiple illumination pathways and angles to provide diverse angle measurement. In certain embodiments, the illumination angles can be decoded with digital processing. As shown in this embodiment, a single collection fiber can be used to maintain the simplicity of detection.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
1. J. S. Schuman, M. R. Hee, C. A. Puliafito, C. Wong, T. Pedut-Kloizman, C. P. Lin, E. Hertzmark, J. A. Izatt, E. A. Swanson, and J. G. Fujimoto, “Quantification of nerve fiber layer thickness in normal and glaucomatous eyes using optical coherence tomography,” Archives of Ophthalmology 113, 586 (1995).
2. R. Pasternack, J. Zheng, and N. N. Boustany, “Detection of mitochondrial fission with orientation dependent optical Fourier filters,” Cytometry Part A (2010).
3. K. Chalut, J. Ostrander, M. Giacomelli, and A. Wax, “Light scattering measurements of subcellular structure provide noninvasive early detection of chemotherapy-induced apoptosis,” Cancer Res. (2009).
4. W. Ju, Q. Liu, K. Kim, J. G. Crowston, J. D. Lindsey, N. Agarwal, M. H. Ellisman, G. A. Perkins, and R. N. Weinreb, “Elevated hydrostatic pressure triggers mitochondrial fission and decreases cellular ATP in differentiated RGC-5 cells,” Invest. Ophthalmol. Vis. Sci. (2007).
5. J. Dwelle, S. Liu, B. Wang, A. McElroy, D. Ho, M. K. Markey, T. Milner, and H. G. Rylander III, “Thickness, phase retardation, birefringence, and reflectance of the retinal nerve fiber layer in normal and glaucomatous non-human primates,” Investigative ophthalmology & visual science (2012).
6. S. Liu, B. Wang, B. Yin, T. E. Milner, M. K. Markey, S. J. McKinnon, and H. G. R. III, “Retinal nerve fiber layer reflectance for early glaucoma diagnosis,” American Journal of Ophthalmology Under review (2012).
7. X. Huang, Y. Zhou, W. Kong, and R. W. Knighton, “Reflectance Decrease Prior to Thickness Change of the Retinal Nerve Fiber Layer in Glaucomatous Retinas,” Invest. Ophthalmol. Vis. Sci. (2011).
8. A. Desjardins, B. Vakoc, W. Oh, S. Motaghiannezam, G. Tearney, and B. Bouma, “Angle-resolved optical coherence tomography with sequential angular selectivity for speckle reduction,” Optics Express 15, 6200-6209 (2007).
9. A. Desjardins, B. Vakoc, G. Tearney, and B. Bouma, “Speckle reduction in OCT using massively-parallel detection and frequency-domain ranging,” Optics Express 14, 4736 (2006).
10. N. Iftimia, B. E. Bouma, and G. J. Tearney, “Speckle reduction in optical coherence tomography by “path length encoded” angular compounding,” Journal of biomedical optics 8, 260-263 (2003).
11. B. Elmaanaoui, B. Wang, J. C. Dwelle, A. B. McElroy, S. S. Liu, H. G. Rylander, and T. E. Milner, “Birefringence measurement of the retinal nerve fiber layer by swept source polarization sensitive optical coherence tomography,” Optics Express 19, 10252-10268 (2011).
12. M. D. Feldman, T. E. Milner, J. H. Oh, E. Kim, K. Kumar, C. Condit, R. Grant, N. Kemp, J. Kim, and S. Chen, “OCT using spectrally resolved bandwidth,” (Give patent number).
13. G. M. Pocock, “The Relationship between Retinal Ganglion Cell Axon Constituents and Birefringence in the Primate Retinal Nerve Fiber Layer,” in Biomedical Engineering (The University of Texas at Austin, Austin, 2009).
14. U.S. Pat. No. 8,174,702

1. An apparatus comprising:
an optical coherence tomography light source configured to emit a first wavelength;
a polarizer configured to polarize the first wavelength;
a splitter configured to direct the first wavelength emitted from the coherence tomography light source to a reference path and to a sample path; and
a pathlength multiplexing element, wherein the pathlength multiplexing element comprises a plurality of regions configured to direct the first wavelength at a plurality of angles in the sample path.

2. The apparatus of claim 1 wherein the plurality of regions of the pathlength multiplexing element comprise a first radial region and a second radial region.

3. The apparatus of claim 1 wherein the plurality of regions of the pathlength multiplexing element comprise a first azimuthal region and a second azimuthal region.

4. The apparatus of claim 1 wherein the plurality of regions comprise four azimuthal regions.

5. The apparatus of claim 1 wherein the plurality of regions comprise six azimuthal regions.

6. The apparatus of claim 1 wherein the plurality of regions of the pathlength multiplexing element comprise a first radial region, a second radial region, a first azimuthal region and a second azimuthal region.

7. The apparatus of claim 1 wherein the plurality of regions comprise a first region configured as an aperture formed in a second region comprising glass.

8. The apparatus of claim 1 wherein the plurality of regions each comprise different refractive indices.

9. The apparatus of claim 1 wherein the optical coherence tomography light source is a swept-source laser.

10. The apparatus of claim 9 wherein the swept-source laser is configured to produce a wavelength of approximately 1060 nm with a 100 kHz sweep rate.

11. The apparatus of claim 1 wherein the sample path is configured to direct the first wavelength toward a retina.

12. The apparatus of claim 1 wherein the sample path is configured to direct the first wavelength toward vascular tissue.

13. The apparatus of claim 1 further comprising an electro-optic modulator between the polarizer and the pathlength multiplexing element.

14. The apparatus of claim 1 wherein the reference path comprises a first polarization beam splitter for a horizontal channel and a second polarization beam splitter for a vertical channel.

15. A method of imaging a sample site, the method comprising:
emitting a first wavelength from an optical coherence tomography light source;
directing the first wavelength to a reference path and a photodetector;
directing the first wavelength to a sample path and through a pathlength multiplexing element to a sample site, wherein:
the first wavelength passes through a first region of the pathlength multiplexing element and is directed to the sample site at a first angle; and
the first wavelength passes through a second region of the pathlength multiplexing element and is directed to the sample site at a second angle;
reflecting the first wavelength from the sample site and through the first region of the pathlength multiplexing element to the photodetector;
reflecting the first wavelength from the sample site and through the second region of the pathlength multiplexing element to the photodetector; and
performing a comparison of the first wavelength reflected from the sample site to the first wavelength from the reference path.

16. The method of claim 15 further comprising determining the size of an object in the sample site based on the comparison of the first wavelength reflected from the sample site to the first wavelength from the reference path.

17. The method of claim 16 wherein the object in the sample site is a sub-cellular structure.

18. The method of claim 16 wherein the object in the sample site comprises mitochondria.

19. The method of claim 15 wherein the sample site comprises a retina.

20. The method of claim 15 wherein the sample site comprises a vascular wall.
